(19) United States
(12) Patent Application Publication
(10) Pub. No.: US 2009/0069279 A1
(43) Pub. Date: Mar. 12, 2009

(54) INVERT EMULSIONS CONTAINING DHEA

(75) Inventors: Fanny ASTRUC, Mougins le Haut (FR); Sandrine Orsoni, Mandelieu (FR); Laurent Fredon, Roquefort les Pins (FR); Jean-Thierry Simonnet, Paris (FR); Pascal Richart, Paris (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, S.N.C., Biot (FR)

(21) Appl. No.: 12/168,356

(22) Filed: Jul. 7, 2008

Related U.S. Application Data
(63) Continuation of application No. 10/767,814, filed on Jan. 30, 2004, now abandoned.
Application No. 10/767,814 is a continuation of application No. PCT/FR02/02569, filed on Jul. 18, 2002.

(30) Foreign Application Priority Data
Aug. 2, 2001 (FR) 01/10398

(51) Int. Cl. A61K 31/56

(52) U.S. Cl. 514/178

(57) ABSTRACT

Stable, recrystallization-resistant invert emulsions, suited, e.g., for preventing/treating the signs of chronological or actinic skin aging and for preventing/treating atrophy of the skin or mucous membranes, comprise a cosmetically/therapeutically effective amount of DHEA and/or chemical and/or biological precursor or derivative thereof, such invert emulsions also comprising a glycolic or hydroglycolic dispersed hydrophilic phase, a lipophilic continuous phase and an emulsifier having an HLB ranging from 2 to 7.

### CROSS-REFERENCE TO EARLIER APPLICATIONS

This application is a continuation of copending U.S. application Ser. No. 10/767,814, filed Jan. 30, 2004, which is a continuation of PCT/FR 02/02569, filed Jul. 18, 2002 and designating the United States (published in the French language on Feb. 13, 2003 as WO 03/011243 A1; the title and abstract were also published in English), claiming priority of FR 01/10398, filed Aug. 2, 2001, all hereby expressly incorporated by reference and all assigned to the assignee hereof.

### BACKGROUND OF THE INVENTION

1. Technical Field of the Invention

The invention relates to novel invert emulsion-type compositions containing DHEA and/or its chemical and/or biological precursors or derivatives thereof, and to the applications therefor in the fields of cosmetics and dermatology.

2. Description of Background and/or Related and/or Prior Art

Human skin consists of two compartments, namely a deep compartment, the dermis, and a top compartment, the epidermis.

The dermis provides the epidermis with a solid support. It is also its feeder component. It is mainly composed of fibroblasts and an extracellular matrix which is itself mainly composed of collagen, elastin and a substance, called ground substance. Leukocytes, mastocytes or tissue macrophages are also present therein. It also contains blood vessels and nerve fibers.

The epidermis is in contact with the external environment. Its role consists in protecting the body from dehydration and from external attacks, whether they are chemical, mechanical, physical or infectious.

The natural human epidermis is mainly composed of three types of cell which are the keratinocytes, which are highly predominant, the melanocytes and the Langerhans cells. Each of these cell types contributes, by its specific functions, to the essential role played in the body by the skin.

The cells constituting the epidermis are delimited by a lipid domain. The epidermal lipids are mainly synthesized in the living epidermis. They are essentially composed of phospholipids, sphingolipids, cholesterol, free fatty acids, triglycerides, esters of cholesterol and alkanes. During cell differentiation, the phospholipids, whose role consists in producing the fluid structure of the cell membranes of the living layers of the epidermis, are gradually replaced by a mixture which is predominantly composed of fatty acids, cholesterol and sphingolipids, which are essential constituents of the horny layer of the epidermis (stratum corneum).

The lipids of the intercorneocyte cement of the skin, and in particular the ceramides, are organized into lamellar bilayers or sheets and participate in the cohesion of the stratum corneum in order to maintain the integrity of the barrier and its protective, antipenetration and in particular anti-irritation role.

It can be understood why activation of the metabolism in the living cells of the epidermis, or an increase in cell proliferation in the living layers, will result in an increase in the epidermal content of phospholipids (sphingomyelin/phosphatidylinositol or phospholipids of the membranes, respectively) and will result in an increase in the size or in the number of living cells, that is to say in a thickening of the epidermis.

This physiological activation will thus make it possible to prevent or to combat the signs of chronological or actinic aging and certain skin pathologies.

Indeed, it is known that during chronobiological aging, in particular during the menopause, atrophy of the epidermis is observed which results from a general slowing of cellular metabolism and which is partly responsible for the appearance of wrinkles and fine lines. Atrophy of the epidermis has also been identified as one of the histological signs of photoaging (Gilchrest B. A., Skin and Aging Processes, 1989, CRC Press).

This process is also present in other mucous membranes, in particular during vulvar or vaginal atrophy.

It can therefore be understood why it is important to have a means for facilitating cell multiplication or metabolism, in particular of the living cells of the epidermis, for preventing or combating atrophy of the epidermis and thus giving the skin a young appearance again.

DHEA or dehydroepiandrosterone, also known as 3-beta-hydroxyandrosteron-5-en-17-one or dehydroisoandrosterone or trans-dehydroandrosterone or prasterone, is a natural steroid essentially produced by the adrenocortical glands.

Exogenous DHEA, administered by the topical or oral route, is known for its capacity to promote keratinization of the epidermis (JP-0-7,196,467) and to treat dry skins by increasing the endogenous production and the secretion of sebum and by thereby reinforcing the barrier effect of the skin (U.S. Pat. No. 4,496,556). There has also been described in U.S. Pat. No. 5,843,932 the use of DHEA for remedying the atrophy of the dermis by inhibiting the loss of collagen and of connective tissue. Finally, the assignee hereof has demonstrated the capacity of DHEA to combat the weathered appearance of the skin (FR-2,803,513), to modulate the pigmentation of the skin and of the hair (EP-1,092,423) and to combat the atrophy of the epidermis. These properties of DHEA make it a candidate of choice as anti-aging active agent.

However, DHEA exhibits the difficulty of being very sparingly soluble in commonly used cosmetic or pharmaceutical solvents such as water, polar or apolar oils.

It is indeed known that DHEA is only soluble with difficulty in aqueous media, which limits its formulation in cosmetic or dermatological compositions applied by the topical or oral route. It thus has a tendency to recrystallize.

DHEA indeed has several polymorphic forms of which the type and the distribution can be poorly controlled, these being dependent on environmental conditions and the manner of preparing the active ingredient (Chang et al., J. Pharm. Sci., 84: 1169-1179 (1995)). There are between three and five anhydrous polymorphic forms and at least three hydrated forms. These polymorphic forms can only be distinguished by analytical techniques such as X ray diffraction, infrared spectroscopy and DSC (differential scanning calorimetry) (WO 00/54763). Depending on the source of supply of DHEA, there may be a variable polymorphic distribution of the raw material, which can potentially cause significant variations in therapeutic bioavailability and in efficacy.

The result is a loss of efficacy and an uncertainty as regards the more or less large dose of DHEA present in these compositions, depending on the degree of recrystallization, which runs counter to the desired objective. In addition, this recrystallization can modify the overall stability of these compositions and their appearance, which can put the user off these compositions.

Moreover, controlling the stability of a particulate dispersion can prove difficult to achieve.

In Table 1 are various examples showing the low solubility of DHEA in a lipophilic phase:

TABLE 1

| INCI NAME | Solubility (% w/w) |
|---|---|
| Caprylic/capric triglycerides | 1.77% |
| Sesame oil | 1.40% |
| Isopropyl palmitate | 1.37% |
| Mineral oil | 1.00% |
| Octyl palmitate | 1.00% |
| Cetearyl isononanoate | 0.83% |
| Dimethicone | 0.17% |
| Squalane | 0.10% |
| Cyclomethicone | 0.04% |

These maximum solubilities of DHEA were measured after stirring for 12 h with a magnetic stirrer bar, at room temperature, with an excess of active ingredient in the excipient to be analyzed. The suspension is then filtered (1.2 μm) and then the filtrate is assayed by HPLC.

Above the concentration measured, there is recrystallization of the DHEA at room temperature.

Despite this, the galenic form most commonly used today is the oil-in-water emulsion in which the DHEA is present in the lipophilic phase. However, this solution remains unsatisfactory because to achieve an objective of an active agent concentration having a quantifiable therapeutic efficacy, very high concentrations of solvent oils would be required, leading to products which are without doubt not very pleasant to use, while having limited DHEA concentration.

The production of an invert emulsion (the expression invert emulsion is understood to mean an emulsion of the type: hydrophilic phase dispersed in a lipophilic phase) as an alternative was not evident to one skilled in this art given the known difficulties of solubility of DHEA in water.

The use of other hydrophilic solubilizers such as propylene glycol was also not natural to one skilled in this art given that the high concentrations required were not favorable for good stability and an acceptable cosmetic feel.

The obtaining of good tolerance with solubilizers such as propylene glycol was also not evident because skin intolerance phenomena had been shown in humans, for example in healthy humans (Motoyoshi et al., Cosmet. and toiletries, 99, 83-89, 1984) where propylene glycol appeared as an irritant at high concentrations, but only under occlusion.

Finally, a use of a water-in-oil type composition had been mentioned in the prior art. Thus, FR-2,777,194 describes a water-in-oil type cosmetic or dermatological composition containing 10% to 50% of a C₂₀ to C₄₀ branched saturated liquid hydrocarbon or of a mixture of such hydrocarbons, 1% to 47% of a natural phospholipid or of a mixture of natural phospholipids and 50% to 80% of water. However, although DHEA is generically mentioned in an extensive list of active compounds, no concrete data is provided. In addition to the absence of an effective embodiment in this prior art, persons skilled in the art were not inclined to follow this formulation route given the high percentage of water taught in FR-2,777,194 (50% to 80%) and the low solubility of DHEA in water (maximum solubility of 0.02 mg/ml), in which DHEA precipitates very rapidly.

In addition, this prior art is mainly centered on the use of phospholipids and not on the production of formulations which can be used for complex derivatives such as DHEA, and/or its chemical and/or biological precursors or derivatives.

Too, phospholipids exhibit limited chemical stability in relation to the phenomena of oxidation, which does not encourage those skilled in this art to rely on this document in their search for stable formulations based on DHEA or analogs.

A need therefore exists for compositions making it possible to respond to one or more of the following aspects: have good stability to cold and to heat, in particular as regards maintaining the size of the globules and the absence of phase separation, have good resistance to the phenomena of oxidation, allow good stability and bioavailability of DHEA and/or its chemical and/or biological precursors or derivatives, exhibit good skin tolerance. It would also be useful to provide compositions allowing a high dispersed volume fraction. It is moreover useful for the preparation of such compositions to exhibit an advantageous mode of preparation.

### SUMMARY OF THE INVENTION

It has now surprisingly and unexpectedly been determined that a glycol-type formulation in oil makes it possible to avoid or ameliorate the various problems linked to the aspects mentioned above, while making it possible in particular to have good stability of the composition per se but also to allow good stability and bioavailability of DHEA and/or its chemical and/or biological precursors or derivatives which it contains. The compositions according to the invention also have the advantage of exhibiting good skin tolerance and allowing a high dispersed volume fraction.

It has now been discovered, in particular, that it is possible to use the good solubility of DHEA at high levels in hydrophilic glycols (demonstrated by the assignee hereof) to obtain a stable formulation while avoiding recrystallization of the active ingredient.

The present invention therefore features compositions containing DHEA and/or its chemical and/or biological precursors or derivatives, the composition comprising an invert emulsion containing a glycolic or hydroglycolic dispersed hydrophilic phase, a lipophilic continuous phase and an emulsifier having an HLB of between 2 and 7.

The term HLB is understood to mean the Hydrophilic/Lipophilic Balance (HLB) which corresponds to the balance between the size and the strength of the hydrophilic group and the size and the strength of the lipophilic group of the emulsifier.

The invention also makes it possible to dispense with the problems caused by the polymorphism of DHEA and also to obtain good bioavailability of the active agent in the skin, DHEA being used in solubilized form.

The expression solubilized form is understood to mean a dispersion in the molecular state in a liquid, no crystallization of the active agent being visible with the naked eye not even under a cross-polarization optical microscope.

The formulation of DHEA, and/or its chemical and/or biological precursors or derivatives, solubilized in a glycolic or hydroglycolic phase, as an invert emulsion thus makes it possible, surprisingly, to dispense with the problems of recrystallization, by ripening or aging (Kabalnov et al., J. Colloid and Interface Science, 118 (1987) 590-597) thereof.

### DETAILED DESCRIPTION OF BEST MODE AND SPECIFIC/PREFERRED EMBODIMENTS OF THE INVENTION

The present invention therefore features invert emulsions, containing a glycolic or hydroglycolic hydrophilic phase, which are essentially perfectly stable (size of the globules and viscosity), even in a high dispersed volume fraction, showing no significant recrystallization of the DHEA, and/or its chemical and/or biological precursors or derivatives.

The expression DHEA precursors is understood to mean its immediate biological precursors or substrates as well as its chemical precursors. Examples of biological precursors are Δ⁵-pregnenolone and 17α-hydroxypregnenolone, without this list being limiting. Examples of chemical precursors are sapogenins such as diosgenin (or spirost-5-en-3-beta-ol), hecogenin, hecogenin acetate, smilagenin and sarsapogenin, and natural extracts containing them, in particular fenugreek and extracts of Dioscorea such as the root of wild yam, without this list being limiting.

The expression DHEA derivatives is understood to mean both its metabolic derivatives and its chemical derivatives. As metabolic derivatives, there may be mentioned, in particular, 7-α-OH-DHEA, 7-β-OH-DHEA, 7-keto-DHEA, Δ⁵-androstene-3,17-diol and Δ⁴-androstene-3,17-dione, without this list being limiting.

As chemical derivatives, there may also be mentioned the esters, such as the esters of hydroxycarboxylic acids and of DHEA which are described in U.S. Pat. No. 5,736,537 or the other esters such as DHEA salicylate, acetate, valerate and enanthate.

Other chemical derivatives of DHEA suitable for carrying out the present invention are the derivatives of formula (1):

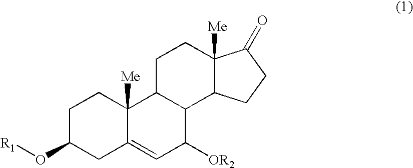

in which:
R₁ and R₂ are independently chosen from:

a linear, branched or cyclic, saturated or unsaturated, C₁-C₁₂ alkyl group which may optionally contain one or more heteroatoms, and may be optionally substituted with one or more groups chosen from —OR′ and/or —SR′ and/or —COOR′ and/or —NR′R′ and/or halogen and/or sulfate and/or phosphate and/or aryl and/or heterocycle, it being possible for said heterocycle to be advantageously chosen from an indole, a pyrimidine, a piperidine, a morpholine, a pyran, a furan, a piperazine, a pyridine;

an alkylcarbonyl group, in which the C₁-C₂₄ alkyl part is linear, branched or cyclic, saturated or unsaturated, and is optionally substituted with one or more groups chosen from —OR′ and/or —SR′ and/or —COOR′ and/or —NR′R′ and/or halogen and/or sulfate and/or phosphate and/or aryl and/or heterocycle, it being possible for said heterocycle to be advantageously chosen from an indole, a pyrimidine, a piperidine, a morpholine, a pyran, a furan, a piperazine, a pyridine;

an arylcarbonyl, preferably a phenylcarbonyl, group or an arylalkylcarbonyl, preferably a benzylcarbonyl, group optionally substituted with one or more groups —OR′ and/or —SR′ and/or —COOR′ and/or —NR′R′ and/or halogen and/or aryl and/or heterocycle;

a group O═P(OH)OR′;

a group (O)₂SOR′;

a trialkylsilyl (SiR₁₃) group in which the 3 groups R′ may be identical or different;

a carbonyloxyalkyl (R′OCO) group;

a carbonylaminealkyl (R′NHCO) group; in which R′ is chosen from a hydrogen atom, a linear, branched or cyclic, saturated or unsaturated, C₁-C₁₂, preferably C₁-C₆, alkyl group which may optionally contain one or more heteroatoms, optionally functionalized with one or more groups —OR″, —COOR″, halogen, —NR″R″; or with an aryl, preferably a phenyl, group optionally functionalized with one or more groups —OR″, —COOR″, halogen, or —NR″R″;

R″ representing a hydrogen atom, a linear, branched or cyclic, saturated or unsaturated, preferably C₁-C₆, alkyl chain,
it being understood that in each of the groups —NR′R′ and —NR″R″, the substituents R′, respectively R″, are identical or different.

Among the derivatives of formula (1), there may be mentioned, in particular, the diesters of 7-OH-DHEA and more particularly 3-O-acetyl-7-benzoyl-oxydehydroepiandrosterone which is, in particular, available from the company GATTEFOSSE under the trade name 3-acetoxy-7-benzoate DHEA.

The composition according to the invention is preferably suitable for topical application to the skin, the superficial body growths and/or the mucous membranes. It generally contains a physiologically acceptable medium and a sufficient quantity of DHEA-based compound to obtain the desired effect. The proportion by weight of DHEA, and/or its chemical and/or biological precursors or derivatives, relative to the total weight of the composition may thus be between 0.001% and 20% (weight/weight), for example between 0.1% and 20%, in particular between 0.2% and 10%, in particular between 0.2% and 4%, for example between 0.2% and 2%.

The glycols to be considered in the present invention may be defined as alkylene or polyalkylene glycols. As nonlimiting examples, there may be mentioned alkylene and polyalkylene glycols (C₁ to C₆) such as ethylene glycol, polyethylene glycol (2 to 20 monomers), propylene glycol, dipropylene glycol, butylene glycol, pentylene glycol, hexylene glycol. They may be oxyethylenated or otherwise (2 to 50 EO). Those preferred according to the invention are hexylene glycol, propylene glycol and dipropylene glycol.

The glycols which can be used according to the invention will advantageously have, as solubility parameter, a δp of less than 10, it being understood that the 3 Hansen solubility parameters: δd, δp and δh characterize, for a given constituent, the energies corresponding respectively to the dispersive, polar and hydrogen bond-type interactions which exist between the molecules of this constituent, δp characterizing more particularly the Debye forces of interaction between dipoles and being a function of the number of oxygen atoms in the formula of the given constituent (S. Paint Technology, 30, 195, 1967, “The three dimensional solubility parameter-Key to paint component affinities”).

The volume fraction of the dispersed hydrophilic phase in the emulsion according to the invention ranges from 10% to 90% relative to the total volume of the emulsion. It may be exclusively glycolic or hydroglycolic, it being understood that the DHEA, and/or its chemical and/or biological precursors or derivatives, are preferably solubilized therein. The volume proportion of glycols (relative to the total volume of the dispersed phase) is between 10% and 100%, for example between 30% and 100%, in particular between 60% and 100%, and preferably between 80% and 100%.

For a cosmetic regime or regimen, there will preferably be used between 30% and 50% of glycols (proportion relative to the total volume of the dispersed phase).

It is also possible to characterize a preferred embodiment of the invention with reference to the water activity (a_{w}) of the hydrophilic phase in the composition according to the invention.

The invention thus also relates, in particular, to a composition as defined above, characterized in that the water activity a_{w} of the hydrophilic phase is less than 0.85.

The water activity a_{w} of a water-containing medium is the ratio of the vapor pressure of water in the product “P_{H2O} product” to the vapor pressure of pure water “P_{H2O} pure” at the same temperature. It may also be expressed as the ratio of the number of water molecules “N_{H2O}” to the total number of molecules “N_{H2O}+N_{dissolved substances}” which takes into account those of dissolved substances “N_{dissolved substances}”.

It is given by the following formulae:

$${a}_{w} = \frac{{P}_{H20} product}{{P}_{H20} pure} = \frac{{N}_{H20}}{{N}_{H20} + {N}_{dissoved substances}}$$

It is possible to use various methods to measure the water activity aw. The most common is the manometric method by which the vapor pressure is directly measured.

Conventionally, a cosmetic or dermatological composition has a water activity which is around 0.95 to 0.99. A water activity of less than 0.85 represents a substantial decrease.

The emulsifiers (surfactants) are natural or synthetic substances consisting of a hydrophilic or polar part and a lipophilic or apolar part. They are amphiphilic molecules since they have a double polarity. Emulsifiers are characterized by their HLB; if the HLB is high, the hydrophilic fraction is predominant; if the HLB is low, the lipophilic part predominates.

Among these emulsifiers are preferably polymeric emulsifiers which are characterized by a high molar mass and a nonlinear structure which allows greater anchorage at the water/oil interface than that obtained with monomeric-type emulsifiers.

The emulsifiers which it is possible to use according to the invention, alone or as a mixture, are those which make it possible to make invert emulsions and which have an HLB of less than 7.

In general, the preferred emulsifiers are organopolysiloxanes such as:

E1) polyalkyl methicone copolyols (optionally crosslinked oxyalkylenated polyalkyl methylsiloxane) containing:

linear or branched, saturated or unsaturated, C₆ to C₂₀ alkyl chains

a polyoxyethylenated unit of 1 to 50 EO (ethylene oxide)

and/or

a polyoxypropylenated unit of 1 to 50 PO (propylene oxide)

E2) oxyalkylenated polyalkyl dimethyl methylsiloxane containing: linear or branched, saturated or unsaturated, C₆ to C₂₀ alkyl chains a polyoxyethylenated unit of 1 to 50 EO

and/or

a polyoxypropylenated unit of 1 to 50 PO.

The organopolysiloxanes of the composition of the invention contain in particular one or more oxyalkylenated, and in particular oxyethylenated (EO), groups, for example from 1 to 40 oxyalkylenated units, preferably from 1 to 20, even better from 10 to 20, more preferably from 12 to 20 and even better from 12 to 18 oxyalkylenated units, which can form polyoxyalkylenated, and in particular polyoxyethylenated, chains. These groups may be pendent or at the chain end. The silicon atoms carrying these groups are advantageously from about 1 to 10, and even better from 1 to 6, in number. The silicone structure forming the polymeric backbone of the organopolysiloxane with (an) oxyalkylenated group(s) is advantageously a polydimethylsiloxane (PDMS) structure of which a portion of the methyl groups is optionally substituted by C₂ to C₃₀ and preferably C₈ to C₂₄ and even better from C₁₀ to C₂₀ alkyl or phenyl groups, either at the chain end or pendent.

Advantageously, there will therefore be used as E1 or E2 type emulsifiers, silicone emulsifiers such as alkyl dimethicone copolyols such as Abil EM-90, or the mixture of dimethicone copolyol and cyclomethicone, sold by the company Dow Corning under the name 3225C Formulation Aid, lauryl methicone copolyol sold under the name Emulsifier 10 by Dow Corning, or mixtures based on a silicone polymer such as cetyl dimethicone copolyol with polyglyceryl-4 isostearate and hexyl laurate sold under the name Abil WE09 by the company Goldschmidt, Abil EM 97 from Goldsclunidt (dimethicone copolyol & cyclomethicone), Wacker SPG 128 VP from Wacker (cyclomethicone and octyldimethicone methoxyglycosyl), or Silwax WD-IS (dimethicone copolyol isostearate).

E3) Mono- or polyalkyl ester siloxanes, for example Silwax S from Lambent (dimethiconol stearate),

E4) alkoxylated carboxylic acid esters such as polyhydroxylated alkyl esters of PEG, for example Arlacel P 135 from Uniqema (PEG-30 dipolyhydroxystearate).

Emulsifiers which will be preferably used have an HLB of between 2 and 7, preferably a siliconized W/O emulsifier having an HLB of between 2 and 7, preferably a polymeric siliconized W/O emulsifier having an HLB of between 2 and 7.

The invert emulsion of the invention may be a variant which is advantageously prepared and stabilized with emulsifiers or the following combinations having an emulsifying character.

1) The combination of an oxyalkylenated crosslinked elastomeric organopolysiloxane and a crosslinked and at least partially neutralized poly(2-acrylamido-2-methylpropanesulfonic acid) polymer.

In particular, the organopolysiloxane with (an) oxyalkylenated group(s) may contain one or more silicone backbones linked to each other by one or more oxyalkylenated, and preferably oxyethylenated, groups as defined above, or by one or more alkylenated groups, the number of alkylenated groups ranging from 1 to 30, and preferably from 1 to 20. Preferably, it contains at least two polymeric backbones linked to each other.

Advantageously, the silicone backbone(s) of the organopolysiloxanes of the compositions according to the invention contain from 26 to 80 silicon atoms. The elastomeric organopolysiloxanes used in the compositions in accordance with the invention are partially or completely crosslinked and have a three-dimensional structure. Incorporated into a lipophilic phase, they are converted, according to the level of lipophilic phase used, from a product with a spongy appearance, when they are used in the presence of low contents of lipophilic phase, to a homogeneous gel in the presence of higher quantities of lipophilic phase. The gelling of the lipophilic phase by these elastomers may be total or partial. These elastomeric organopolysiloxanes may be provided in powdered form, the particles constituting this powder having a size generally ranging from 0.1 to 500 μm, preferably from 3 to 200 μm, and even better from 3 to 50 μm, it being possible for them to be spherical, flat or amorphous with, preferably, a spherical shape. They may also be provided in the form of an anhydrous gel containing the elastomeric organopolysiloxane dispersed in an oily phase. The organopolysiloxanes of the composition of the invention are for example that marketed under the reference KSG 21 by the company Shin Etsu or the product of example 3 (example of synthesis) of U.S. Pat. No. 5,412,004.

2) Alkyl ester and alkyl ether derivatives of polyglycerol, esters of polyethylene glycols, alkyl esters of sorbitan, metal salts of fatty acids, such as diglyceryl diisostearate and sorbitan monooleate (Span 80 from Uniqema).

3) The oligomers and polymers consisting of an apolar polyolefin part and at least one polar part. They can have a block or comb type structure.

The apolar polyolefin part comprises at least 40 carbon atoms, and preferably from 60 to 700 carbon atoms. It is important that this part contains at least 40 carbon atoms in order to achieve the aim of the invention. If there are less than 40 carbon atoms, a satisfactory stable system is not obtained. This apolar part may be chosen from polyolefins such as oligomers, polymers and/or copolymers of ethylene, ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins are hydrogenated or otherwise.

Moreover, the polyolefin-derived oligomers or polymers used in the compositions of the invention contain at least one polar part. This polar part confers amphiphilic properties on the polyolefin derivatives. Thus, these oligomers or polymers lower the interfacial tension (water/oil interfacial tension, that is to say between the aqueous phase and the oily phase) by at least 10 mN/m when they are present at a concentration of 0.01% by weight relative to the total weight of the oily phase. For example, the polyolefin with a succinic ending described below and marketed under the name L2724 by the company Lubrizol, at a concentration of 0.01% by weight relative to the total weight of the oily phase, lowers the interfacial tension by 15 mN/m at the interface of an aqueous phase consisting of a 1% aqueous MgSO₄ solution, and of an oily phase containing a mixture of oils (isohexadecane/hydrogenated polyisobutene/volatile silicone in an 8/6/4 ratio).

The polar part of the oligomeric or polymeric emulsifiers of the invention may be anionic, cationic, nonionic, zwitterionic or amphiphilic. It consists for example of polyalkylene glycols or polyalkylene imines, or alternatively carboxylic acids or diacids, anhydrides thereof or derivatives thereof, and mixtures thereof. Oligomeric or polymeric emulsifiers with a polar carboxylic acid part may for example be derived from the reaction between a polyolefin and at least one carboxylic acid or anhydride chosen from the group comprising maleic acid, maleic anhydride, fumaric acid, itaconic acid, citraconic acid, mesaconic acid and aconitic acid. Preferably, the polar part consists of succinic acid or anhydride, ester or amide derivatives thereof, the corresponding salts of alkali metal, alkaline-earth metal or organic ions, or alternatively of polyoxyethylene.

The polyoxyethylene-derived emulsifiers may be chosen for example from polyisoprene-polyoxyethylene diblock polymers, poly(ethylene-co-propylene)-polyoxyethylene polymers and mixtures thereof. These polymers are described in the publication by Allgaier, Poppe, Willner, Richter (Macromolecules, 1997, vol. 30, p. 1582-1586).

The succinic acid or anhydride-derived emulsifiers may be chosen in particular from the polyolefin derivatives of succinic acid or anhydride described in U.S. Pat. Nos. 4,234,435, 4,708,753, 5,129,972, 4,931,110, GB-2,156,799 and U.S. Pat. No. 4,919,179 incorporated here for reference. The polyolefin part may consist for example of hydrogenated or nonhydrogenated polyisobutylene having a molecular weight ranging from 400 to 5 000. In the polyisobutylene with a succinic ending thus obtained, the succinic part may be esterified, amidated or in salt form, that is to say that it may be modified with alcohols, amines, alkanolamines or polyols, or may be in the form of salts of an alkali or alkaline-earth metal, of ammonium or of an organic base such as the salts of diethanolamine and of triethanolamine. The polyolefins with an esterified or amidated succinic ending are products of the reaction of (a) a polyolefin with a succinic ending, and of (b) an amine or an alcohol, to form an amide or an ester. The term “amine” used here comprises all types of amines including alkanolamines. They may be for example primary, secondary or tertiary monoamines, it being possible for these amines to be saturated or unsaturated, aliphatic, cycloaliphatic, aromatic or heterocyclic. Moreover, the alcohols may be mono- or polyalcohols. The monoalcohols comprise primary, secondary or tertiary aliphatic alcohols, and phenols. The polyalcohols may be chosen for example from aliphatic, cycloaliphatic, aromatic and heterocyclic polyalcohols. The polyolefins with a modified succinic ending (esterified or amidated) and their method of preparation are described in particular in U.S. Pat. No. 4,708,753 which is incorporated here for reference.

As polyolefins with a succinic ending, there may be mentioned in particular polyisobutylenes with a modified succinic ending, such as the products marketed under the names L2724 and L2721 by the company Lubrizol. Another example of a polymeric emulsifier which can be used in the invention is the product of the reaction of maleic anhydride with polyisobutylene, such as the product marketed under the name Glissopal SA by the company BASF. The quantity of emulsifying oligomer(s) or polymer(s) in the composition of the invention may range for example from 0.1% to 10% by weight of active substance, preferably from 0.5% to 5% by weight, and even better from 1% to 3% by weight relative to the total weight of the composition. It is possible to use one or more oligomers or polymers derived from polyolefins. According to a preferred embodiment of the invention, the oligomers or polymers derived from polyolefins are the only emulsifiers used in the composition according to the invention.

4) The alkyl polyglycosides having an HLB of less than 7, combined with an oxyalkylenated polydimethylsiloxane. The alkyl chain of the alkyl polyglycoside preferably comprises from 14 to 22 carbon atoms and may be in particular an unsaturated linear chain or a branched chain, and more particularly the oleyl or isostearyl chain. The alkyl polyglycosides used according to the present invention may be more particularly represented by the following general formula (I):

R—O(G)x (I)

in which R represents an unsaturated linear alkyl radical or a branched alkyl radical, containing from 14 to 24 carbon atoms, G represents a reduced sugar containing from 5 to 6 carbon atoms, and x denotes a value ranging from 1 to 15. Preferred alkyl polyglycosides according to the present invention are compounds of formula (I) in which R denotes more particularly an alkyl radical containing from 16 to 22 carbon atoms, G denotes glucose, fructose or galactose, x is a value ranging from 1 to 4 and more particularly from 1 to 2. According to the invention, in formula (I), R is an unsaturated linear alkyl radical (that is to say an alkylene radical) or a branched alkyl radical. The unsaturated alkyl radical may comprise one or more ethylenic unsaturations, and in particular one or two ethylenic unsaturations. According to a preferred embodiment of the invention, the radical R contains 18 carbon atoms and denotes in particular an oleyl radical (unsaturated C₁₈ radical) or an isostearyl radical (saturated C₁₈ radical), G denotes glucose and x is a value ranging from 1 to 2. The alkyl polyglycoside used in the emulsion of the invention is preferably chosen from the group comprising isostearyl glucoside, oleyl glucoside and mixtures thereof. The oxyalkylenated polydimethylsiloxanes considered are those described in paragraph E1 above.

The composition according to the invention will contain in particular, expressed as a percentage by weight, from 0.5% to 8% of emulsifier, for example from 0.5% to 5%, preferably between 3% and 5%, relative to the total weight of the composition.

Moreover, advantageously, to improve the stability of the dispersion, it is possible to supplement the principal emulsifier(s) described above with one or more coemulsifiers having an HLB of greater than 6. The (coemulsifier/emulsifier) ratio will be advantageously less than 1.5, and preferably less than 0.75.

By way of example, there may be mentioned:

polyoxyethylenated or nonpolyoxyethylenated alkyl or polyalkyl esters of sorbitan with between 1 and 5 branched or unbranched, saturated or unsaturated alkyl chains between C₁₀ and C₂₀, and with 0 to 40 EO (for example: sorbitan monolaurate 20 EO or sorbitan monooleate 200 EO (Tween 80 from Uniqema))

polyoxyethylenated alkyl or polyalkyl ethers or esters with between 1 and 5 branched or unbranched, saturated or unsaturated alkyl chains between C₁₀ and C₂₀, and with 0 to 40 EO (ceteareth-20 (Eumulgin B2 from Cognis), or steareth (Brij 78) 20 EO)

ethoxylated and esterified alkyl or polyalkyl mono- or polyglucosides with between 1 and 5 branched or unbranched, saturated or unsaturated alkyl chains between C₆ and C₂₀, and from 1 to 10 glucose units (for example PEG-20 methyl glucose sesquistearate (SSE-20 glucamate from Amerchol))

alkyl or polyalkyl esters or ethers of polyglycerol with between 1 and 5 branched or unbranched, saturated or unsaturated alkyl chains between C₁₀ and C₂₀, and from 1 to 8 glycerol units (for example polyglyceryl-4 isostearate or PEG-8 stearate (Myrj 45)).

Finally, it is possible to add, advantageously to the dispersed phase from 0% to 10% by weight, relative to the total weight of the formulation, of a cosolvent for DHEA having an evaporation temperature of less than 100° C., preferably linear or branched hydrophilic C₁ to C₄ alcohols such as ethanol and isopropanol.

Advantageously, the preparation of the emulsions according to the invention was found to require little mechanical or thermal energy compared with the preparations of other invert emulsions already known.

In a known manner, the composition of the invention may also contain the usual adjuvants in the cosmetic and dermatological fields, such as hydrophilic or lipophilic gelling agents, humectants such as glycerin and sorbitol, hydrophilic or lipophilic active agents, fatty phase thickeners, preservatives, antioxidants, electrolytes, solvents, perfumes, fillers, screening agents, pigments, odor absorbers and coloring matter. The quantities of these various adjuvants are those conventionally used in the fields considered, and are for example from 0.01% to 20% of the total weight of the composition. These adjuvants, depending on their nature, may be introduced into the lipophilic phase or into the hydrophilic phase. These adjuvants, and their concentrations, should be such that they do not adversely affect the cosmetic or dermatological properties of DHEA and/or its chemical and/or biological precursors or derivatives, in the composition according to the invention.

As fatty substances which can be used for the continuous lipophilic phase in the emulsions according to the invention, it is possible to use oils, and in particular mineral oils (liquid paraffin), oils of plant origin (avocado oil, soybean oil), oils of animal origin (lanolin), synthetic oils (perhydrosqualene), silicone oils (cyclomethicone) and fluorinated oils (perfluoropolyethers). It is also possible to use, as fatty substances, fatty alcohols such as cetyl alcohol, fatty acids, waxes and gums, and in particular silicone gums.

Preferably, nonoxidizable fatty substances are used for the oils of the continuous lipophilic phase, which are preferably chosen from those of the silicone type, those of the ester type or those of the mineral type.

Preferably, the lipophilic phase is not a solvent for DHEA.

As hydrophilic gelling agents, there may be mentioned in particular carboxyvinyl polymers (carbomer), acrylic copolymers such as acrylate/alkyl acrylate copolymers, polyacrylamides, polysaccharides, natural gums and clays, and as lipophilic gelling agents, there may be mentioned modified clays such as bentones, metal salts of fatty acids and hydrophobic silica.

As active agents, it is possible to use in particular isoflavonoids, metalloproteinase inhibitors, carotenoids, antiglycation compounds, NO-synthase inhibitors, vitamins, desquamating agents, compounds increasing the synthesis of glycosaminoglycans, anti-irritant compounds, compounds reducing irritation of neurogenic origin, muscle-relaxing compounds and depigmenting agents.

The compositions according to the invention have a cosmetically acceptable feel, good skin tolerance, stability (the expression stability is understood to mean physical stability, that is to say absence of phase separation and maintenance of the size of the globules and nonrecrystallization of the active agent) to cold (at 4° C.) and to heat (45° C.) over a long period, for example over 2 months, with a stable viscosity.

In particular, the invention also relates to cosmetic or dermatological compositions for topical application to the skin, the superficial body growths and/or the mucous membranes, in the form of an invert emulsion containing a dispersed glycolic or hydroglycolic hydrophilic phase and a lipophilic continuous phase, characterized in that it contains, in a physiologically acceptable medium (that is to say compatible with topical application to the skin, the superficial body growths and/or the mucous membranes), expressed as a percentage by weight:

from 0.001% to 5% of DHEA and/or of its chemical and/or biological precursors or derivatives,

from 30% to 100% of glycols,

from 0.5% to 8% of emulsifier having an HLB of between 2 and 7,

from 0% to 5% of coemulsifier having an HLB greater than 6,

from 0% to 50% of water, for example from 0% to 30% of water.

In a particular embodiment of the invention, the dispersed hydrophilic phase has a water activity of less than 0.85.

The invention also extends to compositions which are triple emulsions of the hydrophilic phase/lipophilic phase/hydrophilic phase type containing an external hydrophilic phase, and a lipophilic phase constituting, with an inner hydrophilic phase, an invert emulsion (termed primary invert emulsion in the context of this triple emulsion) according to the invention.

Advantageously, the present invention relates to a triple emulsion of the hydrophilic phase/lipophilic phase/hydrophilic phase type where the inner hydrophilic phase of the triple emulsion has a water activity value of less than or equal to 0.85, in particular for improving the stability of the active agent present in the inner hydrophilic phase.

According to a particular embodiment of the invention, the water activity value of less than or equal to 0.85 is obtained by incorporating an effective quantity of glycol. The expression effective quantity is understood to mean a sufficient quantity of polyol for obtaining a low water activity value, that is to say a water activity value of less than or equal to 0.85.

According to a particular embodiment of the invention, the primary invert emulsion constitutes from 20% to 35%, and more particularly about 25% by weight of the triple emulsion.

The triple emulsion is prepared in a conventional manner by preparing the primary emulsion and incorporating a defined quantity of primary emulsion into the external hydrophilic phase.

The invention also extends to a triple emulsion of the hydrophilic phase/lipophilic phase/hydrophilic phase type containing an external hydrophilic phase, a lipophilic phase constituting, with an inner hydrophilic phase, an invert emulsion (termed primary invert emulsion in the context of this triple emulsion) according to the invention comprising a gelled external hydrophilic phase containing:

1) at least one emulsifying copolymer consisting of a predominant fraction of a monoolefinically unsaturated C₃-C₆ carboxylic acid monomer or of its anhydride and of a minor fraction of a fatty ester monomer of acrylic acid, and

2) at least one crosslinked poly(acrylamidomethyl propanesulfonic acid).

Moreover, according to a preferred embodiment of the invention, the lipophilic phase of the triple emulsion according to the invention contains at least one silicone oil and/or one silicone emulsifier.

The emulsifying copolymers which can be used in the triple emulsions according to the present invention are prepared by polymerizing a preponderant quantity of a monoolefinically unsaturated carboxylic monomer or its anhydride, at a lower quantity of fatty chain acrylic ester monomer. The expression fatty chain is understood to mean a linear or branched alkyl radical containing from 8 to 30 carbon atoms.

The quantity of carboxylic monomer or its anhydride preferably ranges from 80% to 98% by weight, and more particularly from 90% to 98% by weight, while the acrylic ester monomer is present in quantities ranging from 2% to 20% by weight and more particularly from 1% to 10% by weight, the percentages being calculated relative to the weight of the two monomers.

The preferred carboxylic monomers are chosen from those corresponding to the following formula (I):

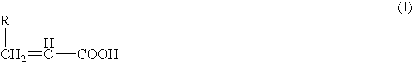

where R denotes hydrogen, a halogen, a hydroxyl group, a lactone group, a lactam group, a cyanogen group (—C═N), a monovalent alkyl group, an aryl group, an alkylaryl group, an aralkyl group or a cycloaliphatic group.

The particularly preferred carboxylic monomers are chosen from acrylic acid, methacrylic acid or mixtures thereof.

The fatty chain acrylic ester monomers are generally chosen from those corresponding to the following formula (II):

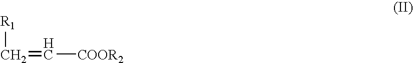

where R₁ is chosen from the group consisting of hydrogen, a methyl radical and an ethyl radical, and R₂ is a C₈-C₃₀ alkyl radical.

The particularly preferred ester monomers are those of which R₁ is hydrogen or a methyl radical and R₂ is a C₁₀-C₂₂ alkyl radical.

The emulsifying copolymers may be optionally crosslinked with the aid of a crosslinking agent used in a quantity ranging from 0.1% to 4%, preferably from 0.2% to 10% by weight relative to the total weight of carboxylic monomers and of acrylic ester monomers. The crosslinking agent is chosen from polymerizable monomers containing a polymerizable CH₂═C— group and at least one other polymerizable group, in which the unsaturated bonds are not conjugated with respect to each other.

The emulsifying copolymers of the invention are described in EP-A-0-268, 164 and are obtained according to the methods of preparation described in this same document.

The particularly preferred emulsifying copolymers are those having a viscosity, measured with a BROOKFIELD viscometer in a solution of water at 2% and at 25° C., of less than or equal to 5,000 cps (5 Pa·s), and more particularly of the order of about 3 000 cps (3 Pa·s).

An acrylate/C₁₀-C₃₀ alkyl acrylate copolymer, and in particular that sold under the name PEMULEN TR 1 by the company GOODRICH, is more particularly used.

The emulsifying copolymer is used in the triple emulsion according to the invention in a concentration ranging for example from 0.05% to 3%, and preferably from 0.1% to 1%, and even better from 0.2% to 0.6% of the total weight of the emulsion.

The invention also covers the use of the novel invert emulsion as described above in cosmetics and in dermatology.

The compositions thus find application in cosmetics, in particular for treating and/or protecting the skin, the mucous membranes or the keratinous fibers, that is to say the hair and the eyelashes.

The compositions according to the invention also find application in a regime or regimen for the prevention and/or the treatment of the signs of chronological or actinic skin aging, and in the treatment of certain pathologies.

The present invention therefore also relates to the cosmetic use of the compositions mentioned above for preventing and/or treating chronological or actinic aging, in particular:

for preventing or reducing the weathered appearance of the skin, and/or

for improving the homogeneity of the color of the skin and/or for lightening the skin and/or brightening up the radiance of the complexion, and/or

for treating wrinkles and fine lines, and/or

for combating flabbiness of the skin, and/or

for combating or preventing atrophy of the skin and of the mucous membranes,

for combating dryness of the skin.

It also relates to the use of these compositions for the cosmetic treatment of the scalp, in particular for preventing or treating canities.

The present invention also relates to the cosmetic use of the compositions according to the invention for attenuating pigmented spots.

The invention moreover extends to the use of a composition according to the invention for manufacturing a pharmaceutical preparation, in particular for manufacturing a pharmaceutical preparation intended for preventing or treating atrophy of the skin or of the mucous membranes, in particular intended for preventing or treating vulvar or vaginal atrophy.

The invention also covers the pharmaceutical preparations and the medicaments obtained from the compositions according to the invention.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

In the compositions below (examples 1 to 6), the proportions of the various constituents are expressed as percentages by weight. They are prepared in the following manner:

Preparation of Phase B1:

The DHEA is solubilized in propylene glycol.

Preparation of Phase B2:

The electrolyte (MgSO₄ or NaCl) is dissolved in water. Add Phases B2 and B3 to Phase B1 and heat to 50° C.

Preparation of Phase A:

The hydrophobic constituents are mixed and heated to 50° C. Phase B is incorporated into Phase A, with moderate mechanical stirring. The DHEA which can be used according to the invention is for example available from the company AKZO NOBEL.

### Example 1

Phase A:

| | |
|---|---|
| Emulsifier 10 (lauryl methicone copolyol) | 5.00% |
| Cyclomethicone | 15.00% |
| Light paraffin oil | 15.00% |
| Ketostearyl alcohol | 3.00% |

Phase B1:

| | |
|---|---|
| Propylene glycol | 19.00% |
| Dipropylene glycol | 32.00% |
| Glycerin | 10.00% |
| DHEA | 1.00% |

### Example 2

Phase A:

| | |
|---|---|
| Emulsifier 10 (lauryl methicone copolyol) | 3.00% |
| Cyclomethicone | 10.00% |
| Paraffin oil | 10.00% |
| Ceteareth-20 | 1.00% |

Phase B1:

| | |
|---|---|
| Propylene glycol | 75.00% |
| DHEA | 1.00% |

### Example 3

Phase A:

| | |
|---|---|
| Emulsifier 10 (lauryl methicone copolyol) | 3.00% |
| Cyclomethicone | 10.00% |
| Cetearyl isononanoate | 7.00% |
| Paraffin oil | 3.00% |
| Ceteareth-20 | 1.00% |

Phase B1:

| | |
|---|---|
| Propylene glycol | 58.00% |
| DHEA | 2.00% |

Phase B2:

| | |
|---|---|
| Water | 10.00% |
| MgSO₄ | 1.00% |

Phase B3:

| | |
|---|---|
| Ethanol | 5.00% |

### Example 4

Phase A:

| | |
|---|---|
| Emulsifier 10 (lauryl methicone copolyol) | 3.00% |
| Cyclomethicone | 15.00% |
| C₁₂-C₁₅ alkyl benzoate | 15.00% |

Phase B1:

| | |
|---|---|
| Propylene glycol | 56.00% |
| DHEA | 1.00% |

Phase B2:

| | |
|---|---|
| Water | 10.00% |

### Example 5

Phase A:

| | |
|---|---|
| Dimethicone copolyol and cyclomethicone | 3.00% |
| Cyclomethicone | 10.00% |
| Cetearyl isononanoate | 7.00% |
| Paraffin oil | 3.00% |
| Ceteareth-20 | 1.00% |
| Zinc stearate | 1.00% |

Phase B1:

| | |
|---|---|
| Propylene glycol | 48.00% |
| DHEA | 1.00% |

Phase B2:

| | |
|---|---|
| Water | 20.00% |
| NaCl | 1.00% |

Phase B3:

| | |
|---|---|
| Ethanol rectapur | 5.00% |

### Example 6

Phase A:

| | |
|---|---|
| Alkyl methicone copolyol | 3.00% |
| Cyclomethicone | 10.00% |
| Cetearyl isononanoate | 7.00% |
| Paraffin oil | 3.00% |
| Ceteareth-20 | 1.00% |

Phase B1:

| | |
|---|---|
| Propylene glycol | 49.30% |
| DHEA | 1.00% |

Phase B2:

| | |
|---|---|
| Water | 20.00% |
| MgSO₄ | 0.70% |

Phase B3:

| | |
|---|---|
| Ethanol | 5.00% |

Rheological Data for the Formula of Example 6:

A HAAKE VT 510 rheometer with an SVDIN measuring rotor was used. The rheograms were produced at 25° C. by varying the shear rate with time, and by measuring the stress. The results obtained are assembled in the Table 2 below:

TABLE 2

| | Yield point | |
|---|---|---|
| | (τ0)in Pa at room | Yield point |
| | temperature | (τ0)in Pa at 45° |
| T = 0 | 61 | / |
| T = 1 month | 52 | not performed |
| T = 2 months | 52 | 48 |

The expression yield point (τ0) is understood to mean the force necessary (minimum shear stress) to overcome the forces of cohesion, of the Van der Waals type and to cause the flow. These data indicate that the viscosity of the product is stable for 2 months at room temperature and at 45° C.

### Example 7

Phase A:

| | |
|---|---|
| Alkyl methicone copolyol | 3.00% |
| Cyclomethicone | 6.00% |
| Cetearyl isononanoate | 7.00% |
| Paraffin oil | 3.00% |
| Ceteareth-20 | 1.00% |
| B.H.T. | 0.10% |

Phase B:

| | |
|---|---|
| Propylene glycol | 58.40% |
| DHEA | 1.50% |

Phase C:

| | |
|---|---|
| Water | 14.00% |
| Electrolytes | 1.00% |
| Ethanol | 5.00% |

Preparation of Phase B:

The DHEA is solubilized in propylene glycol at 55° C.

Preparation of Phase A:

The hydrophobic constituents are mixed and heated to 50° C.

Phase B is incorporated into Phase A, with moderate mechanical stirring at 50° C.

Preparation of Phase C:

The electrolyte is dissolved in water. The ethanol is then incorporated.

This phase is introduced at room temperature into the emulsion, with moderate stirring.

Rheological Data for the Formula of Example 7:

In the same manner as for example 6, the following data are obtained:

| | Yield point (τ0) in Pa at room |
|---|---|
| | temperature |
| T = 0 | 74 |
| T = 3 months | 69 |

Data on the Chemical Stability of DHEA in the Finished Product:

| | Quantity of DHEA in the finished |
|---|---|
| | product in % |
| T = 0 | 102.0 |
| T = 1 month at 55° | 100.8 |
| T = 2 months at 55° | 99.9 |
| T = 3 months at room temperature | 100.7 |
| T = 3 months at 55° | 98.8 |

### Example 8

Phase A

| | |
|---|---|
| Mixture based on cetyl dimethicone copolyol | 3.5% |
| with polyglyceryl-4 isostearate and hexyl | |
| laurate (Abil WE 09 from Goldschmidt) | |
| Hydrogenated polyisobutene | 16.5% |
| Dimethicone (MW 250 000) | 4% |

Phase B

| | |
|---|---|
| Dipropylene glycol | 35% |
| Propylene glycol | 24% |
| DHEA | 1% |
| Distilled water | 15% |

The DHEA was solubilized beforehand in dipropylene glycol and the other constituents of Phase B were then added in order to constitute it. Phase B is introduced into Phase A, with paddle or rotor-stator type stirring. The emulsion is made at a temperature of less than 40° C. A stable, slightly viscous composition is then obtained which shows no recrystallization of the DHEA after at least 15 d at 4° C.

### Examples 9 to 16

### Compositions Containing 7-α-OH-DHEA

In a manner similar to that described above, it is possible to prepare the compositions corresponding to examples 1 to 8, but where, in place of DHEA, its metabolic derivative, 7-α-OH-DHEA, is used.

### Examples 17 to 24

### Compositions Containing 7-keto-DHEA

In a manner similar to that described above, it is possible to prepare the compositions corresponding to examples 1 to 8, but where, in place of DHEA, its metabolic derivative, 7-keto-DHEA, is used.

### Example 25

Study of the release and penetration of DHEA in a formulation according to the invention.

Protocol: The in vitro release/penetration of DHEA and/or its chemical and/or biological precursors or derivatives in compositions according to the invention may be evaluated on total human skin.

The formulation tested is applied for 16 hours to diffusion cells made of glass (3 ml; 1 cm²). Nondermatomed total skin was used. The skin was fixed onto a diffusion cell, the dermis being in contact with a physiological saline solution supplemented with 0.25% (w/w) of an emulsifier (receptor liquid). The system was maintained in static mode (no renewal of the receptor liquid as a function of time).

The skin samples from abdominal and/or breast plastic surgery were used. The formulation is applied to these three different skin samples in an amount of 10 mg of formulation per cm². The applications were made without occlusion. The applications being made in duplicate, the formulations were therefore applied 6 times in total.

At the end of the application time, for each diffusion cell, the excess surface is removed, the receptor liquid and the skin are collected. The epidermis (stratum corneum included) is separated from the dermis. For each formulation tested, a complete evaluation of the active ingredient is calculated taking into account the excess and the quantities found in the skin and in the receiving liquid. The concentrations of active ingredient are determined by means of an HPLC assay with an APCI/MS/MS detection (quantification limit: 10 ng·ml⁻¹).

Result: It is shown in particular that the formulation according to the invention makes it possible to find large quantities of DHEA in the skin.

### Example 26

### Measurement of Skin Tolerance

Protocol: Topical applications of the compositions according to the invention are repeated for 2 weeks on the inner face of the right ear of mice (except during the weekend).

The formulations are placed in tubes, these being stored at room temperature. Each tube is identified by a label on which are noted the study number, the product name, the formulation number, the dose and the expiry date.

The animals used are 7-8-week-old inbred female Balb/C Albino mice at the beginning of the study obtained from IFFA CREDO, France, in groups of 8 mice. The animals are kept acclimatized for at least 5 days before the beginning of the study.

The animals are weighed on D1, on entering the study and placed in an individual cage.

20 μl of the compositions to be tested are applied to the inner face of the right ear of the mice with the aid of a Multipette (Eppendorf 4780 set on position 2, provided with its 0.5 ml Eppendorf Combitip).

The treatments are made at the rate of one application per day, 5 days per week for 2 weeks, the duration of the treatment being variable according to the irritation results.

The animals are observed at D1 before the first treatment, and every two days up to the end of the study.

The measurements of ear thickness are carried out with the aid of an oditest and the ear thickness and the clinical observations are noted.

The mean values of the ear thickness per group and the areas under the curves are calculated and represented by corresponding graphs and histograms.

The corresponding statistical analyzes are made on Minitab, the tests used are Mann-withney and sample-t.

Results: The test composition according to the invention and its placebo, when applied under the same conditions, do not exhibit a significant irritation response. It is therefore considered as being nonirritating.

### Example 27

### Measurement of the Activation of Lipogenesis by the Formulations According to the Invention

The study is carried out on female Syrian hamsters. In these animals, the sebaceous glands situated on the inner face of the ears exhibit characteristics similar to those of the face in humans (number, structure, composition of the sebum).

Protocol: The animals are kept in individual cages during the entire duration of the study and have access to water and to food permanently (Hamster RJ: AURA (TOPS Han). The temperature of the rooms is 22+/−2° C. with a humidity of 55+/−15%. The manipulations performed on the animals are in agreement with the current legislation on the use and the protection of laboratory animals. The hamsters receive a daily dose of the formulations to be tested, of placebos, negative controls or positive controls, by the topical route for 10 days on the inner face of the right ear. At the end of the treatment and after collecting the sample, the cartilage of the right and the left ears is removed with the aid of a scalpel and skin biopsies having a diameter of 8 mm are taken for analyzes of the lipid composition and for histology.

Analyzes: The various study groups are composed of 10 animals: 5 animals are intended for the study of the lipid composition of the sebaceous glands by thin-layer chromatography; 5 animals are intended for histological analysis.

Analysis of the lipid composition: the skin collected from each animal is maintained in 12-well culture plates containing DMEM medium supplemented with bovine fetal serum (10%), in the presence of antibiotics and antifungals. Radiolabeled acetate is added to the cultures and these are kept in an incubator at 37° C. for a period of 6 hours. The biopsies are then recovered, rinsed with PBS. The radiolabeled lipids are extracted in various mixtures of solvents according to the Blye & Dyer procedure and taken up in 400 μl of a dichloromethane/methanol (2/1) mixture. The samples are then deposited per group of 20 on 10×20 silica plates for HPTLC using a depositing robot (Camag-ATSIV). Samples of standards are also deposited in parallel and then the plates are developed in a triple migration system. After carbonization with copper sulfate, they are dried and exposed for 16 hours in cassettes containing a “phosphorimager” film. The various lipids are identified by comparing with the standard. The lipid fractions are then analyzed and quantified with the aid of the TINA software and the results are processed with the aid of the Excel software.

Histological analysis: the skin biopsies are kept in cassettes for histology between two foam buffers impregnated with 10% paraformaldehyde and then embedded in paraffin. They are cut and stained with the aid of a hemalum-phloxine-saffron mixture. The measurements of the thickness of the epidermis are then carried out.

Results: It appears that the formulation according to the invention allows the induction of lipogenesis in a dose-dependent manner on the treated skin while preserving good skin tolerance.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. An invert emulsion comprising a cosmetically/therapeutically effective amount of DHEA and/or chemical and/or biological precursor or derivative thereof, said invert emulsion also comprising a glycolic or hydroglycolic dispersed hydrophilic phase, a lipophilic continuous phase and a silicone emulsifier having an HLB ranging from 2 to 7, said DHEA and/or chemical and/or biological precursor or derivative thereof being solubilized in the glycolic or hydroglycolic dispersed hydrophilic phase, said invert emulsion being stable and recrystallization-resistant.

2. (canceled)

3. The stable invert emulsion as defined by claim 1, said silicone emulsifier comprising lauryl methicone copolyol, cetyl dimethicone copolyol, a mixture of dimethicone copolyol and cyclomethicone, or a mixture of cetyl dimethicone copolyol with polyglyceryl-4 isostearate and hexyl laurate.

4. The stable invert emulsion as defined by claim 1, further comprising a coemulsifier having an HLB greater than 6.

5. The stable invert emulsion as defined by claim 4, said coemulsifier comprising ceteareth-20.

6. The stable invert emulsion as defined by claim 1, the volume proportion of glycol/hydroglycol, relative to the total volume of the dispersed phase, ranging from 10% to 90%.

7. The stable invert emulsion as defined by claim 1, said dispersed hydrophilic phase comprising at least one glycol selected from the group consisting of propylene glycol, hexylene glycol and dipropylene glycol.

8. The stable invert emulsion as defined by claim 1, the water activity a_{w} of said dispersed hydrophilic phase being less than 0.85.

9. (canceled)

10. A triple invert emulsion comprising a cosmetically/therapeutically effective amount of DHEA and/or chemical and/or biological precursor or derivative thereof, said triple invert emulsion also comprising a glycolic or hydroglycolic dispersed hydrophilic phase, a lipophilic continuous phase and an emulsifier having an HLB ranging from 2 to 7, and said triple invert emulsion being of hydrophilic phase/lipophilic phase/hydrophilic phase type which comprises an external hydrophilic phase and a lipophilic phase constituting, with an inner hydrophilic phase, an invert emulsion, said triple invert emulsion being stable and recrysallization-resistant.

11. The stable invert emulsion as defined by claim 1, further comprising one or more active agents selected from the group consisting of isoflavonoids, metalloproteinase inhibitors, carotenoids, antiglycation compounds, NO-synthase inhibitors, vitamins, desquamating agents, compounds increasing the synthesis of glycosaminoglycans, anti-irritant compounds, compounds reducing irritation of neurogenic origin, muscle-relaxing compounds and depigmenting agents.

12. The stable invert emulsion as defined by claim 1, comprising a cosmetically/therapeutically effective amount of DHEA.

13. The stable invert emulsion as defined by claim 1, comprising a cosmetically/therapeutically effective amount of a precursor of DHEA.

14. The stable invert emulsion as defined by claim 13, said DHEA precursor being selected from the group consisting of Δ⁵-pregnenolone, 17 α-hydroxypregnenolone, diosgenin, hecogenin, hecogenin acetate, smilagenin and sarsapogenin.

15. The stable invert emulsion as defined by claim 1, comprising a cosmetically/therapeutically effective amount of DHEA derivative.

16. The stable invert emulsion as defined by claim 15, said DHEA derivative being selected from the group consisting of 7-α-OH-DHEA, 7-β-OH-DHEA, 7-keto-DHEA, 5-androstene-3,17-diol and 4-androstene-3,17-dione.

17. The stable invert emulsion as defined by claim 15, said DHEA derivative having the following structural formula (1): 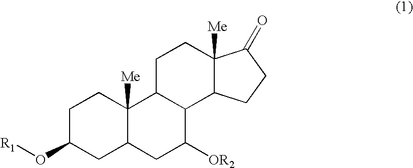
in which R₁ and R₂ are, independently, a linear, branched or cyclic, saturated or unsaturated, C₁-C₁₂ alkyl radical which may optionally contain one or more heteroatoms, and may be optionally substituted with one or more groups selected from among —OR′ and/or —SR′ and/or —COOR′ and/or —NR′R′ and/or halogen and/or sulfate and/or phosphate and/or aryl and/or heterocycle, with the proviso that said heterocycle is an indole, a pyrimidine, a piperidine, a morpholine, a pyran, a furan, a piperazine, a pyridine; an alkylcarbonyl radical, in which the C₁-C₂₄ alkyl moiety is linear, branched or cyclic, saturated or unsaturated, and is optionally substituted with one or more groups selected from among —OR′ and/or —SR′ and/or —COOR′ and/or —NR′R′ and/or halogen and/or sulfate and/or phosphate and/or aryl and/or heterocycle, with the proviso that said heterocycle is an indole, a pyrimidine, a piperidine, a morpholine, a pyran, a furan, a piperazine, a pyridine; an arylcarbonyl radical or an arylalkylcarbonyl radical optionally substituted with one or more groups —OR′ and/or —SR′ and/or —COOR′ and/or —NR′R′ and/or halogen and/or aryl and/or heterocycle; a group O═P(OH)OR′; a group (O)₂SOR′; a trialkylsilyl (SiR′₃) group in which the 3 groups R′ may be identical or different; a carbonyloxyalkyl (R′OCO) group; a carbonylaminealkyl (R′NHCO) group, in which R′ is a hydrogen atom, a linear, branched or cyclic, saturated or unsaturated, C₁-C₁₂ alkyl radical which may optionally contain one or more heteroatoms, optionally functionalized with one or more groups —OR′, —COOR′, halogen, —NR′R′; or with an aryl radical optionally functionalized with one or more groups —OR′, —COOR′, halogen, or —NR′R′, in which R′ is a hydrogen atom, a linear, branched or cyclic, saturated or unsaturated alkyl radical, with the proviso that in each of the groups —NR′R′ and —NR′R′, the substituents R′ and R′ are identical or different.

18. The stable invert emulsion as defined by claim 15, said DHEA derivative comprising 3-O-acetyl-7-benzoyloxydehydroepiandrosterone.

19. The stable invert emulsion as defined by claim 1, comprising from 0.001% to 20% by weight of said DHEA and/or chemical and/or biological precursor or derivative thereof.

20. The stable invert emulsion as defined by claim 1, comprising from 0.2% to 4% by weight of said DHEA and/or chemical and/or biological precursor or derivative thereof.

21. A topically applicable cosmetic/dermatological composition comprising a stable, recrystallization-resistant invert emulsion as defined by claim 1, formulated into a topically applicable, cosmetically/dermatologically acceptable medium therefor.

22. The cosmetic/dermatological composition as defined by claim 21, comprising from 0.001% to 5% by weight of DHEA and/or chemical and/or biological precursor or derivative thereof; from 30% to 100% by weight of said dispersed hydrophilic phase; and from 0.5% to 8% by weight of said emulsifier having an HLB ranging from 2 to 7.

23. The cosmetic/dermatological composition as defined by claim 22, further comprising up to 5% by weight of a coemulsifier having an HBL greater than 6.

24. The cosmetic/dermatological composition as defined by claim 23, further comprising up to 50% by weight of water.

25. A regime or regimen for preventing or treating chronological or actinic skin aging, comprising administering to an individual in need of such treatment, a stable, recrystallization-resistant invert emulsion as defined by claim 1.

26. A regime or regimen for treating and/or protecting the skin, the mucous membranes or the keratinous fibers, comprising administering to an individual in need of such treatment, a stable, recrystallization-resistant invert emulsion as defined by claim 1.

27. A regime or regimen for preventing or treating canities, comprising administering to an individual in need of such treatment, a stable, recrystallization-resistant invert emulsion as defined by claim 1.

28. A regime or regimen for attenuating pigmented skin spots, comprising administering to an individual in need of such treatment, a stable, recrystallization-resistant invert emulsion as defined by claim 1.

29. A regime or regimen for preventing or treating atrophy of the skin or mucous membranes, comprising administering to an individual in need of such treatment, a stable, recrystallization-resistant invert emulsion as defined by claim 1.

30. A regime or regimen for preventing or treating vulvar or vaginal atrophy, comprising administering to an individual in need of such treatment, a stable, recrystallization-resistant invert emulsion as defined by claim 1.

31. The stable invert emulsion as defined by claim 1, comprising a cosmetically/therapeutically effective amount of DHEA, said invert emulsion also comprising a glycolic dispersed hydrophilic phase comprising propylene glycol, a lipophilic continuous phase and a silicone emulsifier having an HLB ranging from 2 to 7 which comprises alkyl methicone copolyol.
